(19) United States
(12) Patent Application Publication
(10) Pub. No.: US 2020/0132700 A1
(43) Pub. Date: Apr. 30, 2020

(54) METHODS OF PREDICTING POST-TRANSFUSION SURVIVAL OF RED BLOOD CELLS

(71) Applicant: BLOODWORKS, Seattle, WA (US)

(72) Inventors: James Charles Zimring, Seattle, WA (US); Xiaoyun Fu, Kenmore, WA (US)

(73) Assignee: BLOODWORKS, Seattle, WA (US)

(21) Appl. No.: 16/099,090

(22) PCT Filed: May 4, 2017
(86) PCT No.: PCT/US17/31135

§ 371 Date: Nov. 5, 2018

Related U.S. Application Data
(60) Provisional application No. 62/332,310, filed on May 5, 2016.

(51) Int. Cl. G01N 33/80; G01N 33/92; A61K 35/18; A61M 1/02

(52) U.S. Cl. CPC G01N 33/80; G01N 33/92; G01N 2800/245; A61M 1/0272; G01N 2405/04; A61K 35/18

(57) ABSTRACT

Methods for predicting post-transfusion survival of red blood cells are provided herein. In some versions, the methods include measuring a level of lysophospholipids in a unit of red blood cells and predicting the post-transfusion survival of the red blood cells in the unit based on the measured level of lysophospholipids.

### INTRODUCTION

In excess of 15,000,000 units of red blood cells (RBCs) are transfused in the United States each year into an excess of 5,000,000 patients (approximately 1 out of every 65 Americans). Currently, there are 3 quality control measures utilized prior to release of a unit of RBCs: 1) testing negative for the screened pathogens, 2) compatibility with the patient regarding recipient antibodies to donor antigens, and 3) storage history of 4° C. FDA guidelines for RBC storage require that stored RBCs have less than 1% hemolysis and have 75% 24 hour post-transfusion survival, on average for a given storage system. However, it has been appreciated for over forty years that there is tremendous variability in how individual units of RBCs store from different human donors. Even for current blood storage solutions, 24 hour post-transfusion recoveries range from 35% to 100%. It has been further observed that RBC storage is reproducible from donation to donation for a given donor, suggesting a potential genetic component.

### SUMMARY

Methods for predicting post-transfusion survival of red blood cells are provided herein. In some versions, the methods include measuring a level of lysophospholipids in a unit of red blood cells and predicting the post-transfusion survival of the red blood cells in the unit based on the measured level of lysophospholipids.

### DETAILED DESCRIPTION

Methods for predicting post-transfusion survival, e.g., historic post-transfusion survival, of red blood cells are provided herein. In some versions, the methods include measuring a level of lysophospholipids in a unit of red blood cells and predicting the post-transfusion survival of the red blood cells in the unit based on the measured level of lysophospholipids.

Before the present invention is described in greater detail, it is to be understood that this invention is not limited to particular embodiments described, as such can, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges can independently be included in the smaller ranges and are also encompassed within the invention, subject to any specifically excluded limit in the stated range. Where the stated range includes one or both of the limits, ranges excluding either or both of those included limits are also included in the invention.

Certain ranges can be presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided can be different from the actual publication dates which can need to be independently confirmed.

It is noted that, as used herein and in the appended claims, the singular forms “a,” “an,” and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Additionally, certain embodiments of the disclosed devices and/or associated methods can be represented by drawings which can be included in this application. Embodiments of the devices and their specific spatial characteristics and/or abilities include those shown or substantially shown in the drawings or which are reasonably inferable from the drawings. Such characteristics include, for example, one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal; distal), and/or numbers (e.g., three surfaces; four surfaces), or any combinations thereof. Such spatial characteristics also include, for example, the lack (e.g., specific absence of) one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal), and/or numbers (e.g., three surfaces), or any combinations thereof.

As will be apparent to those of skill in the art upon reading this disclosure, each of the individual embodiments described and illustrated herein has discrete components and features which can be readily separated from or combined with the features of any of the other several embodiments without departing from the scope or spirit of the present invention. Any recited method can be carried out in the order of events recited or in any other order which is logically possible.

### Methods

The subject methods include predicting how well a RBC unit will circulate post-transfusion by measuring a panel of one or more lysophospholipids. The subject methods provide a diagnostic approach, e.g., an vitro approach, to determine quality of different RBC units of stored blood, on a donor-by-donor basis, and in some versions include directing RBC units of certain quality to certain patients.

The subject disclosure includes methods of predicting post-transfusion survival of red blood cells which include measuring a level of lysophospholipids in a unit of red blood cells. Measuring a level of lysophospholipids in a unit of red blood cells can, in some versions, include measuring the amount of one or more lysophospholipids present in the unit at the time or shortly after, e.g., within 5 minutes or less or 10 minutes or less or 30 minutes or less or 60 minutes or less, the unit is collected from a subject. The methods can also include collecting such a unit from a subject.

The methods can also include predicting the post-transfusion survival, e.g., historic post-transfusion survival, of the red blood cells from a subject in a unit based on the measured level of lysophospholipids. Predicting the post-transfusion survival of the red blood cells in the unit based on the measured level of lysophospholipids can, in various aspects include comparing the measured level to that of a control sample and/or a those of a database of listed amounts, e.g., average lysophospholipids amounts, from samples obtained from one or more. e.g., 5 or more, 10 or more, 100 or more, or 1000 or more, subjects, e.g., normal healthy human subjects.

Predicting the post-transfusion survival of the red blood cells in the unit can include predicting the post-transfusion survival to be 6 hours or more, 12 hours or more, 1 day or more, 2 days or more, 3 days or more, 4 days or more, 5 days or more, 10 days or more, 20 days or more, 30 days or more, 40 days or more, or 42 days or more. Predicting the post-transfusion survival of the red blood cells in the unit can also include predicting the post-transfusion survival to be 6 hours or less, 12 hours or less, 1 day or less, 2 days or less, 3 days or less, 4 days or less, 5 days or less, 10 days or less, 20 days or less, 30 days or less, 40 days or less, or 42 days or less.

In certain embodiments, a subject is a “mammal” or a “mammalian” subject, where these terms are used broadly to describe organisms that are within the class mammalia, including the orders carnivore (e.g., dogs and cats), rodentia (e.g., mice, guinea pigs, and rats), and primates (e.g., humans, chimpanzees, and monkeys). In some embodiments, the subject is a human. The term “humans” can include human subjects of both genders and at any stage of development (e.g., fetal, neonates, infant, juvenile, adolescent, and adult), where in certain embodiments the human subject is a juvenile, adolescent or adult. While the methods described herein can be applied in association with a human subject, it is to be understood that the subject devices and methods can also be applied in association with other subjects, that is, on “non-human subjects.”

In various embodiments, the lysophospholipids include Lysophosphatidylcholine, LysophosphatidylEthanolamine, and/or LysophosphatidylSerine, or any combination thereof. In various embodiments, the lysophospholipids include only Lysophosphatidylcholine. In various embodiments, the lysophospholipids include only LysophosphatidylEthanolamine. In various embodiments, the lysophospholipids include only LysophosphatidylSerine.

Also in some versions, the methods include storing one or more units of red blood cells and/or samples for a length of time, such as storing the units or samples after and/or beginning at a time when the unit or sample is collected and/or an initial marker measurement is taken. Such a length of time can be 6 hours or more, 12 hours or more, 1 day or more, 2 days or more, 3 days or more, 4 days or more, 5 days or more, 10 days or more, 20 days or more, 30 days or more, 40 days or more, or 42 days or more. Such a length of time can also be 6 hours or less, 12 hours or less, 1 day or less, 2 days or less, 3 days or less, 4 days or less, 5 days or less, 10 days or less, 20 days or less, 30 days or less, 40 days or less, or 42 days or less.

The methods also include methods of transfusing an amount of red blood cells to a subject. Such methods can include collecting a unit of red blood cells from a subject, e.g., a human donor: measuring a level of lysophospholipids in the unit of red blood cells; predicting the post-transfusion survival of the red blood cells in the unit based on the measured level of lysophospholipids; and/or storing the unit of red blood cells for a length of time. The methods can also include transfusing an amount of the unit of red blood cells to a subject.

In some versions, the amount of the unit of red blood cells transfused is based on the predicted post-transfusion survival of the red blood cells. In various aspects, the methods include transfusing a smaller amount, e.g., a smaller amount than would be transfused of lower quality blood, of blood when a high post-transfusion survival of the red blood cells is predicted. In various aspects, the methods include transfusing a larger amount e.g., a larger amount than would be transfused of higher quality blood, of blood when a low post-transfusion survival of the red blood cells is predicted.

Also, as used herein, the terms “higher” or “lower” and related terms can refer to higher or lower, respectively, in reference to a control, such as a control red blood cell sample. Such a control sample can be a sample from one or more normal healthy humans. Such a control can also include accepted values associated with samples from one or more normal healthy humans. In some versions, a control is a sample having mean historic 24-hr RBC recoveries.

In some versions, the methods include predicting a longer post-transfusion survival if the concentration of lysophospholipids is high. In some versions, the methods include predicting a longer post-transfusion survival if the concentration of lysophospholipids is low. In some versions, the methods include predicting a shorter post-transfusion survival if the concentration of lysophospholipids is high. In some versions, the methods include predicting a shorter post-transfusion survival if the concentration of lysophospholipids is low.

In some versions of the methods in which blood is transfused, the amount of one or more units of red blood cells transfused is low when the predicted post-transfusion survival is high. In some versions of the methods in which blood is transfused, the amount of one or more units of red blood cells transfused is high when the predicted post-transfusion survival is low.

In some versions, a unit of RBC with a high post-transfusion survival, e.g., a post-transfusion survival predicted based on measurement of a panel of one or more lysophospholipids therein, has a high quality and as such, can be transfused to a subject in a smaller amount than if the unit had a lower quality. Also, a unit of RBC with a low post-transfusion survival, e.g., a post-transfusion survival predicted based on measurement of a panel of one or more lysophospholipids therein, has a low quality and as such, can be transfused to a subject in a larger amount than if the unit had a higher quality.

An “analyte” or “target” refers to a compound to be detected. Such compounds can include small molecules, peptides, proteins, nucleic acids, as well as other chemical entities. In the context of the present invention, an analyte or target will generally correspond to the biochemical compounds disclosed herein, or a reaction product thereof.

The term “biomarker” refers to a molecule (typically small molecule, protein, nucleic acid, carbohydrate, or lipid) that is expressed and/or released from a cell, which is useful for identification or prediction. Such biomarkers are molecules that can be differentially expressed, e.g., overexpressed or underexpressed, or differentially released in response to varying conditions (e.g., storage). In the context of the present invention, this frequently refers to the biochemical compounds disclosed herein, which are elevated in stored versus non-stored platelets, for instance, 1-fold, 2-fold, 3-fold, 4-fold, 5-fold or more in stored platelets versus non-stored platelets.

A “sample” refers to any source which is suspected of containing an analyte or target molecule. Examples of samples which may be tested using the present invention include, but are not limited to, blood, serum, plasma, urine, saliva, cerebrospinal fluid, lymph fluids, tissue and tissue and cell extracts, cell culture supemantants, among others. A sample can be suspended or dissolved in liquid materials such as buffers, extractants, solvents, and the like. In the context of the present application, a sample is generally a stored platelet sample of varying length of storage.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

Samples of platelets stored for various amounts of time are compared to “control” samples which can be freshly drawn platelets or platelets which have been minimally stored. Control samples are assigned a relative analyte amount or activity to which sample values are compared. Relevant levels of analyte elevation occur when the sample amount or activity value relative to the control is 110%, or 150%, or 200-500% (i.e., two to five fold higher relative to the control), or 1000-3000% higher.

As used herein, “PLT storage quality” is defined as the extent of post-transfusion recovery of the stored PLTs: higher recovery is defined as higher quality. Examples of post-transfusion recovery include greater than zero and almost 100% recovery, i.e., recovery of 10%, 20%, 30%, 40%, 50%, 60%, 70%, 80%, 90%, 100%, and all percentages in between.

As used herein. “toxicity” of a PLT unit is defined as any adverse reaction associated with transfusion of a PLT unit, including, but not limited to, fever, inflammation, induction of recipient cytokines, transfusion induced lung injury, and transfusion-related immunomodulation, among others.

As used herein, a PLT unit is less suitable for transfusion if it has lower PLT quality (i.e., post-transfusion survival) or elevated toxicity as compared to other PLT units, e.g., as compared to a control.

As used herein. “transfusion outcome” refers to post-transfusion survival of platelets in the circulation and the presence or absence of toxicity after platelet transfusion.

In some embodiments, the measurement of the markers of the present invention is performed using various mass spectrometry methods. As used herein, the term “mass spectrometry” or “MS” refers to an analytical technique to identify compounds by their mass. MS refers to methods of filtering, detecting, and measuring ions based on their mass-to-charge ratio, or “m/z”. MS technology generally includes (1) ionizing the compounds to form charged compounds; and (2) detecting the molecular weight of the charged compounds and calculating a mass-to-charge ratio. The compounds may be ionized and detected by any suitable means. A “mass spectrometer” generally includes an ionizer and an ion detector. In general, one or more molecules of interest are ionized, and the ions are subsequently introduced into a mass spectrographic instrument where, due to a combination of magnetic and electric fields, the ions follow a path in space that is dependent upon mass (“m”) and charge (“z”). See, e.g., U.S. Pat. No. 6,204,500, entitled “Mass Spectrometry From Surfaces:” U.S. Pat. No. 6,107,623, entitled “Methods and Apparatus for Tandem Mass Spectrometry;” U.S. Pat. No. 6,268,144, entitled “DNA Diagnostics Based On Mass Spectrometry” U.S. Pat. No. 6,124,137, entitled “Surface-Enhanced Photolabile Attachment And Release For Desorption And Detection Of Analytes;” Wright et al., Prostate Cancer and Prostatic Diseases 1999, 2: 264-76; and Merchant and Weinberger, Electrophoresis 2000, 21; 1164-67.

As used herein, the term “gas chromatography” or “GC” refers to chromatography in which the sample mixture is vaporized and injected into a stream of carrier gas (as nitrogen or helium) moving through a column containing a stationary phase composed of a liquid or a particulate solid and is separated into its component compounds according to the affinity of the compounds for the stationary phase.

As used herein, the term “liquid chromatography” or “LC” means a process of selective retardation of one or more components of a fluid solution as the fluid uniformly percolates through a column of a finely divided substance, or through capillary passageways. The retardation results from the distribution of the components of the mixture between one or more stationary phases and the bulk fluid, (i.e., mobile phase), as this fluid moves relative to the stationary phase(s). Examples of “liquid chromatography” include reverse phase liquid chromatography (RPLC), high performance liquid chromatography (HPLC), and turbulent flow liquid chromatography (TFLC) (sometimes known as high turbulence liquid chromatography (HTLC) or high throughput liquid chromatography).

In some embodiments, the present methods are practiced using computer implementation, such as by generating a dataset with a computer. In one embodiment, a computer comprises at least one processor coupled to a chipset. Also coupled to the chipset are a memory, a storage device, a keyboard, a graphics adapter, a pointing device, and a network adapter. A display is coupled to the graphics adapter. In one embodiment, the functionality of the chipset is provided by a memory controller hub and an I/O controller hub. In another embodiment, the memory is coupled directly to the processor instead of the chipset.

The storage device is any device capable of holding data, like a hard drive, compact disk read-only memory (CD-ROM), DVD, or a solid-state memory device. The memory holds instructions and data used by the processor. The pointing device may be a mouse, track ball, or other type of pointing device, and is used in combination with the keyboard to input data into the computer system. The graphics adapter displays images and other information on the display. The network adapter couples the computer system to a local or wide area network.

As is known in the art, a computer can have different and/or other components than those described previously. In addition, the computer can lack certain components. Moreover, the storage device can be local and/or remote from the computer (such as embodied within a storage area network (SAN)).

As is known in the art, the computer is adapted to execute computer program modules for providing functionality described herein. As used herein, the term “module” refers to computer program logic utilized to provide the specified functionality. Thus, a module can be implemented in hardware, firmware, and/or software. In one embodiment, program modules are stored on the storage device, loaded into the memory, and executed by the processor.

Embodiments of the entities described herein can include other and/or different modules than the ones described here. In addition, the functionality attributed to the modules can be performed by other or different modules in other embodiments. Moreover, this description occasionally omits the term “module” for purposes of clarity and convenience.

The subject methods include determining post-transfusion survival of platelets (PLT) prior to transfusion, the methods including the steps of: a) measuring the levels of one or more markers in a PLT sample, wherein the one or more markers include lysophospholipids; and/or b) comparing the level of the one or more markers in the PLT sample with the level of the one or more markers present in a control sample, wherein a higher or lower level of the one or more markers in the PLT sample is indicative of post-transfusion survival of platelets.

The methods also include determining the suitability of a platelet (PLT) unit for use in a blood transfusion, the methods including the steps of: measuring the levels of one or more markers in a PLT sample wherein the one or more markers are or include any one or combination of the lysophospholipids described herein, at for example, the time of donation or shortly thereafter, e.g., 30 min or less. The methods can also include comparing the level of the one or more markers in the PLT sample with the level of the one or more marker present in a control sample, such as a control sample determined to be suitable for use in a blood transfusion, wherein a higher or lower level of the one or more markers in the PLT sample as compared to that of the control sample is indicative of suitability for transfusion. For example, a higher level of markers than the control can indicate that the sample is not suitable for transfusion and the sample is then discarded and not transfused. Also, a lower level of markers than the control can indicate that the sample is suitable for transfusion and the sample is then transfused.

In some aspects, the methods also include determining a higher level of the one or more markers in the stored blood sample compared to the control blood sample that indicates a lower RBC storage quality of the RBCs of the stored blood sample as compared to RBCs of the control blood sample. Such a determination can include predicting a lower post-transfusion survival of the of the RBCs of the stored blood sample than RBCs of the control blood sample, or determining an absence of a higher level of the one or more markers in the stored blood sample compared to the control blood sample. Such a determination, such as determining a higher level of the one or more markers in the stored blood sample compared to the control blood sample, can indicate that a sample is or is not suitable for transfusion and the sample is transfused or discarded accordingly.

The methods can also include determining a lower level of the one or more markers in the stored blood sample compared to the control blood sample that indicates a higher RBC storage quality of the RBCs of the stored blood sample as compared to RBCs of the control blood sample. Such a determination can include predicting a higher post-transfusion survival of the of the RBCs of the stored blood sample than RBCs of the control blood sample, or determining an absence of a lower level of the one or more markers in the stored blood sample compared to the control blood sample. Such a determination, such as determining a lower level of the one or more markers in the stored blood sample compared to the control blood sample, can indicate that a sample is or is not suitable for transfusion and the sample is transfused or discarded accordingly.

In various aspects, the methods also include excluding the stored blood sample from use in the blood transfusion and/or discarding the sample when a lower RBC storage quality of the RBCs of the stored blood sample is indicated as compared to the RBCs of the control blood sample. The methods can also or alternatively include using the stored blood sample in the blood transfusion when an absence of a higher level of the one or more markers in the stored blood sample compared to the control blood sample is determined.

In various embodiments, measuring the levels of one or more markers is performed the time of donation or shortly thereafter, e.g., 10 min or less, or 30 min or less, or 60 min or less. In some versions, it is performed prior to storage of the sample, such as prior to storage of the sample in a refrigerated environment, such as an environment having a temperature less than room temperature, such as 20° C. In some versions, storage of the sample as provided herein includes refrigerated storage of the sample in a refrigerated environment, such as an environment having a temperature of 20° C. or less, such as 18° C. or less, such as 15° C. or less, such as 12.5° C. or less, such as 10° C. or less, such as 5° C. or less, such as 3° C. or less, such as 1° C. or less. In some versions, measurements are performed on the first day of storage.

In some versions, it is performed, or is additionally performed, after the sample has been stored a length of time in the inclusive range of 24 of 48 hours. In some aspects, it is also performed after storing the sample and/or unit for a period of time such as 1 day or less, 15 days or less, 30 days or less, or 45 days or less, or 1 day or more, 15 days or more, 30 days or more, or 45 days or more. In some aspects, it is performed after storing the sample and/or unit for a period of time ranging from 1 to 60 days, such as 1 to 50 days, such as 1 to 30 days, such as 1 to 20 days, such as 1 to 15 days, such as 1 to 10 days.

In various embodiments, measuring the levels of one or more markers is performed in two or more measurements, e.g., 3, 4, 5, 10 or fewer, 15 or fewer, or 20 or fewer measurements. A first measurement can be conducted the time of donation or shortly thereafter, e.g., 10 min or less, or 30 min or less, or 60 min or less, or 1 day or less, or 2 days or less, such as before storage, such as before refrigerated storage. One or more additional measurement such as a second measurement can be performed after storage, such as after refrigerated storage for any of the time periods listed herein. The first and supplemental, e.g., second, measurements can be compared with one another to determine the suitability of the sample for transfusion or each can be evaluated individually to determine the suitability for transfusion.

In various embodiments, performing the measurement does not include measuring a level of a Hydroxyeicosatetraenoic acid (HETE) or Hydroxyoctadecadienoic (HODE) in the sample.

In some aspects, a level of the one or more markers is obtained during the time of storage of the RBC sample. In various embodiments, a level of the one or more markers is obtained by performing mass spectrometry with a spectrometry device. Such a mass spectrometry device can be a gas-chromatography/mass spectrometry (GC/MS) or liquid chromatography-tandem mass spectrometry (LC/MS/MS). In various embodiments, a level of the one or more markers is obtained by performing one or more ELISA assays.

Method according to the subject disclosure also include predicting transfusion outcome, the methods including the steps of: obtaining a dataset associated with a sample of stored platelets, wherein the dataset comprises at least one marker, wherein the dataset comprises data for at least one marker, wherein the one or more markers include lysophospholipids; and/or analyzing the dataset to determine data for the at least one marker, wherein the data is positively correlated or negatively correlated with transfusion outcome if the platelet sample is transfused into a patient.

In various embodiments of the above aspects, the dataset is obtained at the time of collection of the PLT sample. In various embodiments of the above aspects, the dataset is obtained during the time of storage of the PLT sample. In various embodiments of the above aspects, the dataset is obtained by mass spectrometry. In various embodiments of the above aspects, the mass spectrometry is gas-chromatography/mass spectrometry (GC/MS) or liquid chromatography-tandem mass spectrometry (LC/MS/MS).

In various embodiments of the above aspects, the dataset is obtained by enzymatic assay. In various embodiments of the above aspects, the dataset is obtained by ELISA.

In another aspect, disclosed herein is kit for use in predicting transfusion outcome or platelet (PLT) storage quality, the kit comprising: a set of reagents comprising a plurality of reagents for determining from a stored platelet sample data for at least one marker, wherein the one or more markers include lysophospholipids; and instructions for using the plurality of reagents to determine data from the stored platelet sample.

### Utility

RBCs that do not circulate well, after transfusion, will have lower efficacy. This is a potential problem for any person requiring blood, but is a particular problem for patients requiring chronic transfusion, who suffer serious sequelae from iron toxicity and alloimmunization (the formation of antibodies preventing future transfusions). To the extent that units of RBCs can be identified that will have the best post-transfusion circulation, and such units can be directed towards chronic transfusion patients, then there can be a significant decrease in the total number of RBC units used on a patient by patient basis. Even a modest decrease in number of units transfused will mitigate iron toxicity and decrease frequency of alloimmunization. The subject disclosures provides methods by which units of RBCs can be identified that will have the best post-transfusion circulation.

Furthermore, despite extensive study, there is no measurable entity known to predict how an RBC unit will do when transfused. For this reason, there are limited quality control measures (or unit release criteria) regarding quality of RBC units. This is a medical problem since RBCs that survive poorly post-transfusion result in a less efficacious product from the standpoint of RBC replacement. Thus the lack of reliable biochemical markers is an impediment to patient care and identification of such markers has a high clinical utility. The subject disclosure provides methods to predict how an RBC unit will do when transfused and as such, provide improved methods for patient care including lower costs and time-efficient methods.

Furthermore, there is significant donor-to-donor variation in how units of RBCs store. Importantly, no parameters have previously been described that can, based upon their levels at time of collection, predict post-transfusion RBC circulation after storage. Previous untargeted human and animal studies identified lipid metabolism as a pathway of interest in blood storage. As noted above, the subject methods provide ways to predict post-transfusion RBC circulation after storage.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

The methods for quantifying lysophospholipids as set forth herein utilize a liquid-chromatography-tandem mass spec system. However, immunoassays and other methodologies can also be applied for such quantification. The measurements have been applied to 9 units of human RBCs from donors who have previously had autologous RBC storage and post-storage RBC circulation studies (as per 51-chromium), and found significant correlations for specific lysophospholipids, with the following two categories of application:
1. The ability to test RBC units at time of collection, and predict how RBCs will circulate (post-transfusion) after storage.
2. The ability to test RBC after storage (prior to transfusion), and to predict how RBCs will circulate post transfusion.

As such, the subject methods include testing, such as measuring markers as described herein at the time of collection and/or predicting the success of a transfusion of a unit of blood, such as by predicting how RBCs will circulate (post-transfusion) after storage, such as predicting that they will effectively or ineffectively circulate. The methods also include measuring markers as described herein after a time of storage and/or prior to transfusion and predicting the predicting how RBCs will circulate (post-transfusion), such as predicting that they will effectively or ineffectively circulate.

Table 1 is presented below and indicates the current correlations, and p values, that have been generated based upon testing a small number of human donors (n=9) and correlating their levels to historical 51-Cr RBC survival studies in these same volunteers. Additional correlations shown can be statistically significant when extended to a large number of test subjects and with a great degree of variability in RBC storage.

### Methodology

Nine (9) human volunteers were identified, each of whom had previously participated in autologous RBC storage and recovery trials using 51-Cr based labeling. Each volunteer had previously participated in at least two 51-Cr studies, and had shown consistent post-transfusion 24-hr RBC recoveries. New units of leukoreduced RBCs were collected from each donor and were stored for 42 days. Samples were collected from each unit on the first day of storage and just after outdate (43 days). Precise quantitation of a panel of lysophospholipids (LysoPLs) was carried out on supernatants using a liquid chromatography-tandem mass spectrometry approach. Species of Lysophosphatidylcholine (LysoPC), LysophosphatidylEthanolamine (LysoPE), and LysophosphatidylSerine (LysoPS) were each quantified by using corresponding 17:1 LysoPLs as internal standards.

As noted above, the methods include measuring a level of lysophospholipids in a unit of red blood cells. The measured level of lysophospholipids can be any of the values provided in Table 1 or less or any of the values presented in Table 1 or more. The values can also be within any of the inclusive ranges defined by any two values provided in Table 1. In some versions, the methods include measured levels that have a linear correlation between Log concentration and a donor's mean of historical 24-hour recoveries. Also, in some versions of the methods, the methods include producing a p-value less than 0.05 or less than or equal to 0.05 or 0.1 or 1.5. In some versions of the methods, the methods include producing a p-value less than 0.01 or less than or equal to 0.01 or 0.005 or 0.03.

### Results

Levels of both LysoPC 16:0 and 18:0 had a significant negative correlation with historic 24-hr RBC recoveries, as measured on day 1 of storage. LysoPC 16:0, 18:0, LysoPE 18:0 and LysoPS 20:4 had significant negative correlation with historic 24-hr RBC recoveries on day 43 of storage. Other measured LysoPLs had a range of correlations [see Table 1 for data].

Mean historic 24-hr RBC recoveries (range: 68-92%) negatively correlated with levels of both LysoPC 16:0 and 18:0, as measured on day 1 of storage with a false discovery rate (FDR) of 16%. Mean historic 24-hr RBC recoveries also negatively correlated with LysoPC 16:0, 18:0, LysoPE 18:0 and LysoPS 20:4 on day 42 of storage with an FDR of 24%.

The current studies find a correlation of levels of LysoPLs, both at time of collection and after storage, with historic 24-hr RBC recoveries. Significant correlations were identified, despite a very small number of donors (n=9) and a relatively narrow overall range of historic 24-hr recoveries (68-92%).

TABLE 1

| | Day 1 Storage | | Day 43 Storage | |
|---|---|---|---|---|
| | Pearson's | | Pearson's | |
| Metabolites | correlation¹ | p-value | correlation¹ | p-value |
| LysoPC 16:0 | −0.878 | 0.002** | −0.717 | 0.030* |
| LysoPC 18:0 | −0.798 | 0.010** | −0.818 | 0.007** |
| LysoPC 20:4 | −0.448 | 0.227 | −0.451 | 0.223 |
| LysoPC 18:2 | −0.301 | 0.432 | −0.253 | 0.511 |
| LysoPC 18:1 | −0.628 | 0.070 | −0.562 | 0.115 |
| LysoPE 18:0 | −0.545 | 0.129 | −0.741 | 0.022* |
| LysoPE 20:4 | −0.306 | 0.424 | −0.381 | 0.312 |
| LysoPE 18:2 | 0.061 | 0.876 | −0.242 | 0.530 |
| LysoPE 18:1 | 0.003 | 0.995 | −0.274 | 0.476 |
| LysoPS 18:0 | 0.608 | 0.083 | −0.543 | 0.131 |
| LysoPS 20:4 | 0.260 | 0.499 | −0.739 | 0.023** |

¹Linear correlation between Log concentration and donors mean of historical 24-hour recoveries
*p < 0.05 and
**p < 0.01 by Pearson's correlation

### Conclusion

The studies provided herein find a correlation of levels of LysoPLs, both at time of collection and after storage, with historic 24-hr RBC recoveries. Significant correlations were observed despite a small number of donors and a relatively narrow overall range of historic 24-hr recoveries.

Although the foregoing invention has been described in some detail by way of illustration and example for purposes of clarity of understanding, it is readily apparent to those of ordinary skill in the art in light of the teachings of this invention that certain changes and modifications can be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future. i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of predicting post-transfusion survival of red blood cells, the method comprising:
a) measuring a level of lysophospholipids in a unit of red blood cells; and
b) predicting the post-transfusion survival of the red blood cells in the unit based on the measured level of lysophospholipids.

2. The method of claim 1, wherein the lysophospholipids comprise Lysophosphatidylcholine, LysophosphatidylEthanolamine, or LysophosphatidylSerine.

3. The method of claim 2, wherein the lysophospholipids comprise Lysophosphatidylcholine.

4. The method of claim 2, wherein the lysophospholipids comprise LysophosphatidylEthanolamine.

5. The method of claim 2, wherein the lysophospholipids comprise LysophosphatidylSerine.

6. The method of claim 1, wherein method further comprises storing the unit of red blood cells for a length of time.

7. The method of claim 1, wherein the length of time is 30 days or more.

8. The method of claim 1, wherein method further comprises transfusing at least a portion of the unit of red blood cells to a subject.

9. A method of transfusing an amount of red blood cells to a subject, the method comprising:
a) collecting a unit of red blood cells from a human donor;
b) measuring a level of lysophospholipids in the unit of red blood cells;
c) predicting the post-transfusion survival of the red blood cells in the unit based on the measured level of lysophospholipids;
d) storing the unit of red blood cells for a length of time; and
e) transfusing an amount of the unit of red blood cells to a subject.

10. The method of claim 9, wherein the amount of the unit of red blood cells transfused is based on the predicted post-transfusion survival of the red blood cells.

11. The method of claim 9, wherein the amount of the unit of red blood cells transfused is low when the predicted post-transfusion survival is high.

12. The method of claim 9, wherein the length of time is 30 days or more.

13. The method of claim 9, wherein the lysophospholipids comprise Lysophosphatidylcholine, LysophosphatidylEthanolamine, or LysophosphatidylSerine.

14-16. (canceled)

17. A method for determining the suitability of a stored blood sample comprising red blood cells (RBCs) for use in a blood transfusion, the method comprising the steps of:
a) measuring a level of one or more markers in the stored blood sample, wherein the one or more markers comprise lysophospholipids; and
b) comparing the level of the one or more markers in the stored blood sample with a level of the one or more markers present in a control blood sample determined to be suitable for use in a blood transfusion.

18. The method according to claim 13, further comprising determining a higher level of the one or more markers in the stored blood sample compared to the control blood sample that indicates a lower RBC storage quality of the RBCs of the stored blood sample as compared to RBCs of the control blood sample and thereby predicting a lower post-transfusion survival of the of the RBCs of the stored blood sample than RBCs of the control blood sample, or determining an absence of a higher level of the one or more markers in the stored blood sample compared to the control blood sample.

19. The method according to claim 13, further comprising excluding the stored blood sample from use in the blood transfusion when a lower RBC storage quality of the RBCs of the stored blood sample is indicated as compared to the RBCs of the control blood sample, and using the stored blood sample in the blood transfusion when an absence of a higher level of the one or more markers in the stored blood sample compared to the control blood sample is determined.

20. The method according to claim 17, wherein the amount of the unit of red blood cells transfused is based on the predicted post-transfusion survival of the red blood cells.

21. The method according to claim 17, wherein the amount of the unit of red blood cells transfused is low when the predicted post-transfusion survival is high.

22. The method according to claim 17, further comprising storing the sample for 30 days or more.

23. The method according to claim 17, wherein the lysophospholipids comprise Lysophosphatidylcholine, LysophosphatidylEthanolamine, or LysophosphatidylSerine.

24-26. (canceled)
